(19) United States
(12) Patent Application Publication
(10) Pub. No.: US 2007/0043257 A1
(43) Pub. Date: Feb. 22, 2007

(54) Cardiac restraint

(75) Inventor: Frederick Y. Chen, Chestnut Hill, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: The Brigham and Women's Hospital, Inc., Boston, MA (US)

(21) Appl. No.: 11/205,578

(22) Filed: Aug. 16, 2005

(51) Int. Cl. A61F 2/00

(52) U.S. Cl. 600/37

(57) ABSTRACT

A cardiac restraint device includes a central cavity that receives the heart. A chamber surrounding the cavity presses against the surface of the heart when the chamber is filled with fluid. The inner wall defining the chamber is deformable, so that it tends to expand into the cavity and contact the heart. The outer wall of the chamber, or alternatively, a jacket surrounding the chamber, is nondeformable, so that expansive forces of the fluid in the chamber tend to be directed inward against the heart rather than outward.

### CROSS-REFERENCE TO RELATED APPLICATIONS

The present application claims the benefit of and hereby incorporates herein by reference provisional application Ser. No. 60/522,104, filed Aug. 16, 2004.

### BACKGROUND

Heart failure is a health care problem of enormous proportions. There are few effective treatment options. The heart dilates during heart failure. This response by the heart to failure aggravates the failure and results in a relentless, pathologic spiral down.

A variety of devices have been proposed to prevent the heart from dilating. Some work by placing supporting struts through the heart itself. Others involve wrapping the heart in various materials to contain the heart and to prevent expansion. Still others provide fluid pouches that press against various parts of the heart, such as the ventricles, to provide contractile assistance.

### SUMMARY

The present disclosure provides systems and methods for restraining the heart.

In one embodiment, a cardiac restraint device may include a sac having an inner wall, an outer wall, an unpartitioned single chamber enclosed between the walls, and a port in fluid communication with the chamber and accessible from outside the device, to permit instillation of fluid into the chamber and to permit measurement of the fluid pressure. The inner wall may be deformable in response to the instillation of a fluid into the chamber. The outer wall may be so nondeformable as not to expand when a fluid is introduced into the chamber. The device may be so sized and shaped that the inner wall engages the outer surface of the ventricles of a heart when positioned around the heart. The chamber may be so filled with fluid that the inner wall of the device contacts substantially all of the outer surface of the ventricles.

In another embodiment a cardiac restraint device may include a sac having an inner wall, an outer wall in fluid-tight seal with the inner wall, an unpartitioned single chamber enclosed between the walls, and a port in fluid communication with the chamber and accessible from outside the device, to permit instillation of fluid into the chamber and to permit measurement of the fluid pressure. The inner wall may define a cavity so sized and shaped as to receive the left and right ventricles of a heart. The inner wall may also be deformable in response to the instillation of a fluid into the chamber. The outer wall may be so nondeformable as not to expand when a fluid is introduced into the chamber. The device may be so sized and shaped that the inner wall engages the outer surface of the ventricles when positioned around the heart. The inner wall, when the chamber is instilled with fluid, may contact substantially all of the outer surface of the ventricles.

In other embodiments, methods of cardiac restraint may include fitting a device as described above around a heart, so that the left and right ventricles of the heart occupy the cavity, affixing the device to the heart, and instilling fluid into the chamber of the device to exert pressure on the heart, thereby restraining the heart.

### BRIEF DESCRIPTION OF THE DRAWINGS

FIG. 1 depicts an exemplary embodiment of a cardiac restraint device.

FIGS. 2 and 2A depict the exemplary embodiment of FIG. 1 in cross-section.

FIG. 3 depicts another exemplary embodiment of a cardiac restraint device.

FIG. 4 depicts the exemplary embodiment of FIG. 3 in cross-section.

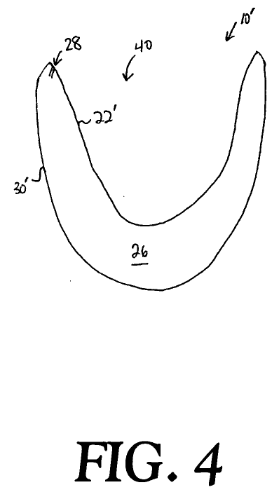

FIG. 5 depicts, in cross-section, an exemplary embodiment of a cardiac restraint device positioned around the ventricles of a heart.

FIG. 6 is a photograph of an embodiment of a cardiac restraint device sac.

FIG. 7 is a photograph of an embodiment of a cardiac restraint device jacket.

FIG. 8 is a photograph of an embodiment of a cardiac restraint device.

FIG. 9 shows the effect of fluid volume on pressure of a cardiac restraint device.

FIG. 10 shows aortic flow, left ventricle (LV) pressure, balloon pressure, and transmural pressure of an exemplary cardiac restraint device, deployed around the ventricles of a heart, over time.

FIG. 11 shows transmural myocardial pressure over the cardiac cycle when an exemplary cardiac restraint device is deployed around the ventricles of a heart. Balloon end diastolic pressures of 0, 3, 5, and 8 mm Hg are shown.

FIG. 12 shows the relationship between average transmural pressure and constraint pressure when an exemplary cardiac restraint device is deployed around the ventricles of a heart.

FIG. 13 shows the relationship between mean arterial pressure and constraint pressure when an exemplary cardiac restraint device is deployed around the ventricles of a heart.

FIG. 14 shows the effect of increasing constraint pressure on transmural myocardial pressure during isometric relaxation, systole filling phase, and diastole filling phase, when an exemplary cardiac restraint device is deployed around the ventricles of a heart.

### DETAILED DESCRIPTION

The disclosed systems and methods facilitate cardiac restraint by surrounding the ventricles of the heart with a device that resists heart dilation. Heart dilation can arise in several pathological conditions of the heart, such as heart failure and valvular disease (examples include mitral valve regurgitation and aortic valve insufficiency). An analysis of a wrap's effect on myocardial mechanics has led to the development of an exemplary cardiac restraint device with appropriate precision with respect to wrap tightness.

The device includes a central cavity that receives the heart. A chamber surrounding the cavity presses against the surface of the heart when the chamber is filled with fluid. The inner wall defining the chamber is deformable, so that it tends to expand inwardly into the cavity and contact the heart. The outer wall of the chamber, or alternatively, a jacket surrounding the chamber, is nondeformable, so that expansive forces of the fluid in the chamber tend to be directed inward against the heart rather than outward.

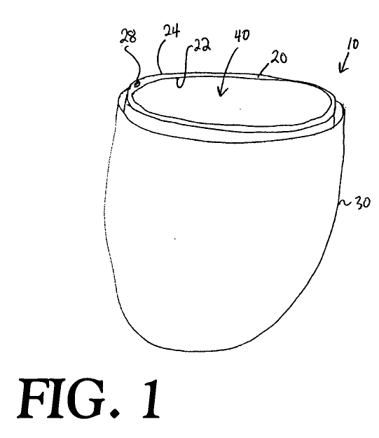

FIG. 1 depicts an exemplary embodiment of a cardiac restraint device 10. In this embodiment, the device includes a sac 20 and a jacket 30 that surrounds the sac. The sac defines a cavity 40 in the center of the device. When used, the device is placed around a heart so that the ventricles of the heart are in the cavity.

The sac 20 includes an inner wall 22 and an outer wall 24. In this embodiment, the inner wall and outer wall define a cavity (not shown) between them. Access to the cavity may be provided by a port 28. A tube or other conduit (not shown) may be attached to the port to facilitate the instillation of fluid into the chamber. The tube may extend to a location in a patient easily reachable through the skin, or out to the patient's skin, to simplify access. The tube may be connected to a portacath or other implantable device that permits percutaneous access. The port 28 may also provide a convenient access point to measure the pressure inside the chamber. As fluid is added to the chamber, the pressure can be monitored and the filling stopped when a desired pressure is reached. In typical applications, the chamber may be pressurized so that it delivers a pressure to the cavity in the range from about 1 mmHg to about 100 mmHg. If the desired pressure is exceeded, fluid can be withdrawn through the port. A patient's heart failure state can be indirectly assessed by measuring the pressure in the chamber. Over time, the pressure can be monitored, and fluid added to or removed from the chamber to adjust the pressure. The chamber, or fluid, pressure may be adjusted so that it is in the range of about 1 mm Hg to about 25 mm Hg at end diastole. The pressure may also be adjusted within subsets of this range, such as about 1 mmHg to about 10 mmHg, about 3 mmHg to about 8 mmHg, about 3 mmHg, about 5 mmHg, about 8 mmHg, about 100 mmHg, about 10 mmHg to about 25 mmHg, about 15 mmHg to about 25 mmHg, about 10 mmHg to about 20 mmHg, about 15 mmHg, about 20 mmHg, or about 20 mmHg to about 25 mmHg (all at end diastole).

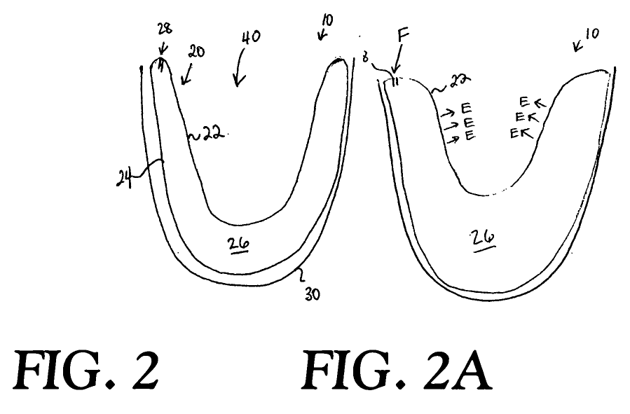

FIG. 2 shows a cross section of the embodiment depicted in FIG. 1. This view shows cavity 26 defined between the inner wall 22 and the outer wall 24 of the sac 20. Although the jacket 30 is shown as detached from the sac 20 for clarity of illustration, in typical use, the jacket will be affixed to the sac (such as by stitching or adhesive). The jacket may be attached to the sac in discrete places, such as at a band around the rim of the jacket; alternatively, the jacket and sac can be affixed more generally, such as by a coating of adhesive on all or substantially all of the inner surface of the jacket and/or outer wall of the sac. In some cases, the jacket and sac may be provided unattached; they may be affixed to one another during a placement procedure.

The outer wall and the inner may be two discrete parts that are joined together to provide the sac a fluid-tight (specifically, liquid-tight) seal. Alternatively, the inner wall and outer wall may simply be two regions of a continuous sac that are defined as a result of the cup-like shape the sac adopts. The sac (or its walls) may be formed of a flexible, deformable material, a wide variety of which are known to be suitable for medical use, such as polyurethane, polyvinylchloride, polyethylene, GORETEX®, polytetrafluoroethylene (PTFE), and others. Materials that resist rupture or leak under the stresses of in vivo implantation are preferred. The device may be composed of non-immunogenic and/or non-inflammatory materials, so that contact of the device does not cause an immunogenic or inflammatory resonse in an individual harboring the device. The device may be composed of inherently non-immunogenic and/or non-inflammatory materials or materials impregnated with the same. In some embodiments, the exposed surfaces of the device may be composed of or coated with such materials. If immunogenic components are used, suitable immuno-suppressive therapy may be necessary. Such immunotherapy is known to those of skill in the art.

In some embodiments, a wide variety of procedures may be used to place the device. Such procedures include open-heart surgery, thoracoscopy, mini-thoracotomy, and subxyphoid approach.

The jacket 30 is typically made of a nondeformable material, such as a fabric, plastic, silicone, and/or rubber. While it may be flexible, a preferred material should not be appreciably expandable, elastic, or otherwise “stretchy.”

In a preferred embodiment, then, a device includes an inner wall that is deformable and a jacket that is nondeformable. FIG. 2A shows the device of FIG. 2 in which the inner wall 22 has been deformed by the addition of fluid F into the chamber. This combination facilitates the inward expansion of the inner wall (the inward expansion is indicated by arrows E), because the deformable inner wall will tend to bulge when the chamber is filled with fluid, while the outer wall will tend to be constrained by the nondeformable jacket. As a result, the expansive forces generated by filling the chamber with fluid will tend to be directed inwardly upon whatever occupies the cavity. In typical use, the ventricles of a heart will occupy the cavity, so the pressurized chamber will expand inward to press against the ventricles, thereby preventing their expansion.

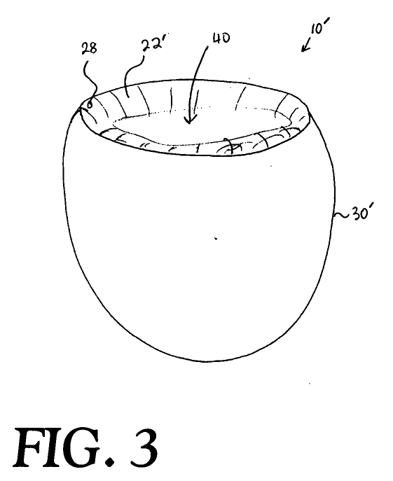

FIGS. 3 (perspective) and 4 (cross section) depict another embodiment of a cardiac restraint device 10′. In this embodiment, the chamber 26 is formed by the inner wall 22′ and the jacket 30′. Instead a full sac being nestled within a jacket, the inner wall is attached directly to the jacket to define the chamber. The inner wall defines a cavity 40 as before, and a port 28 may be provided to permit fluid access to the chamber.

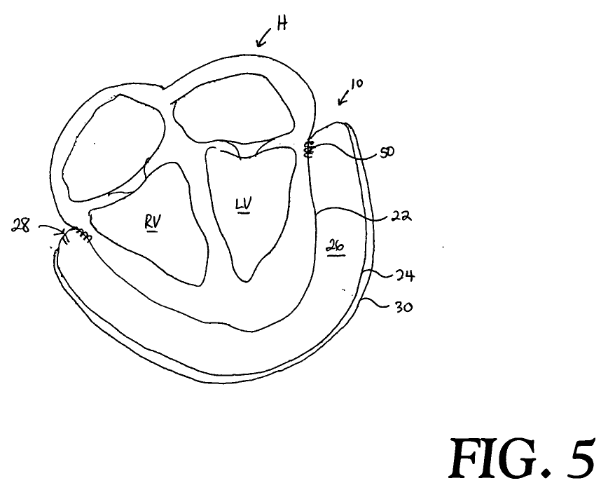

FIG. 5 depicts an exemplary use of a cardiac restraint device as disclosed herein. A cardiac restraint device 10 is shown, in cross section, deployed around the ventricles of a heart H. The device is positioned around the outer surface of the right ventricle RV and left ventricle LV of the heart. Fluid is added into the chamber 26 through port 28. As a result of the pressure created in the chamber, the inner wall 22 expands into the device cavity and presses against the heart. Because the chamber 26 is a single cavity, the inner wall 22 is able to conform to the outer contour of the heart and contact all or substantially all of the outer surface of the ventricles. As a result, the device can press evenly against the surface of the ventricles to provide unbroken support over the ventricular surface. The single-chambered, highly-conforming support helps the heart to retain its shape, because substantially all of the ventricular surface is covered, so the ventricles cannot bulge anywhere. Typically, the inner wall 22 will contact all or substantially all of the ventricular surface when the heart is in diastole. In some instances, the inner wall might not touch the ventricles during systole, because the heart in systole is smaller than in diastole due to contraction.

The inner wall 22 may contact the ventricular surface of the heart directly or indirectly. In some embodiments, the device may be placed directly around the heart (i.e., contacting the epicardium). This can be done by gaining access to the pericardial space and then deploying the device. In other embodiments, the device may be placed around the pericardium (i.e., outside the parietal pericardium) to help prevent adhesion formation. In yet other embodiments, the device may be placed partly around pericardium and partly around the heart.

The device may be attached to the heart so that it can provide restraint over a period of time. For example, the device could be stitched, tacked, or adhered to the heart, any of which is schematically indicated in FIG. 50 as element 50. If the device is designed to cover substantially all of the outer surface of the ventricles, then the device may be attached at the transition between the atria and ventricles.

A cardiac restraint device as disclosed herein may be provided in the form of a kit. The kit may include the device (of any type disclosed herein), a tube connectable to the device's port, a quantity of fluid to be used to fill the chamber, stitches, staples, and/or adhesive. In some cases, the device may be provided preassembled. In other cases, the device may be provided in various stages of disassembly. For example, if the device is of the type that includes a jacket and a sac, the jacket may be separate or preattached. The tube, if an, may be separate of preattached.

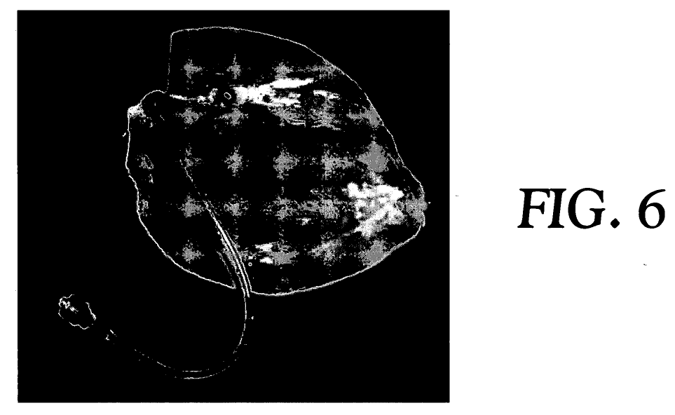

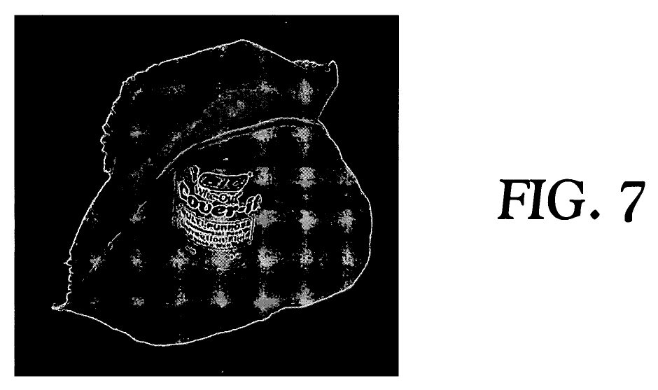

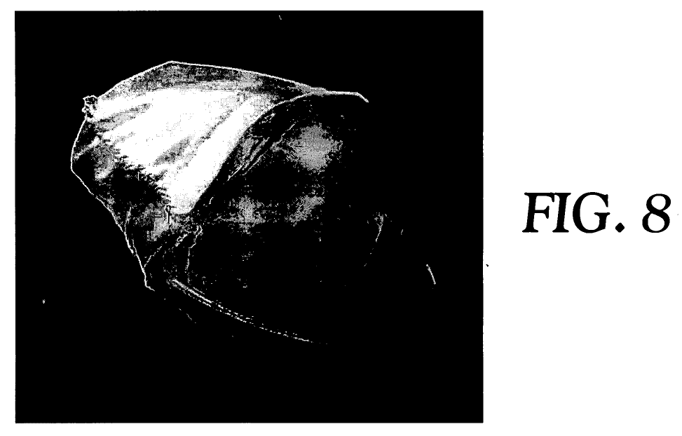

FIGS. 6-8 are photographs of one embodiment of a cardiac restraint device and components thereof. FIG. 6 shows a sac and a tube attached to a port. The pictured device includes a second port, to which nothing is attached. In this case, the sac is made of polyvinylchloride. FIG. 7 depicts a jacket made of fabric. The bottle of correction fluid is present to indicate the cavity and to hold open the jacket for illustrative purposes; it is not part of the device. FIG. 8 depicts an assembled cardiac restraint device, in which the jacket surrounds the sac, with a tube connected to a port in the sac, and the cavity open to receive a heart.

U.S. Pat. Nos. 2,826,193; 4,957,477; 5,702,343; 6,425,856; 6,508,756; 6,547,716; 6,572,534; 6,626,821; and 6,743,169 disclose other kinds of cardiac restraint devices. Each is hereby incorporated herein by this reference.

Exemplification

### EXAMPLE

### Optimization of Constraint Therapy

Wrap tightness and its effect on ventricular mechanics were quantitated so that constraint therapy could be optimized. A cardiac restraint with a fluid-filled balloon (CRAB) device was implanted in eight normal sheep. Four constraint levels, defined as fluid (CRAB) pressure at end-diastole, were applied: 0, ⅓ Pmax, ⅔ Pmax, and Pmax, wherein Pmax is equal to the constraint level that reduces mean arterial pressure by 10 mm Hg. Aortic flow, aortic pressure, left ventricle pressure, and CRAB pressure were measured. CRAB pressure is defined as balloon end diastolic pressure. Repeated-measures ANOVA was used to assess for a change in transmural pressure with increasing ventricular constraint level.

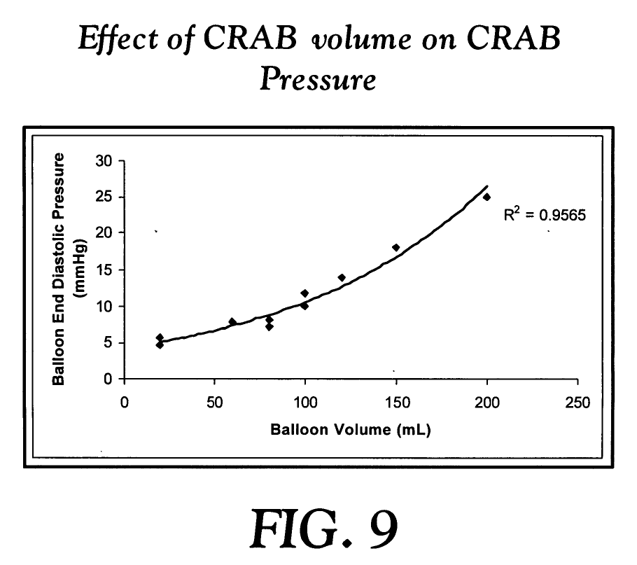

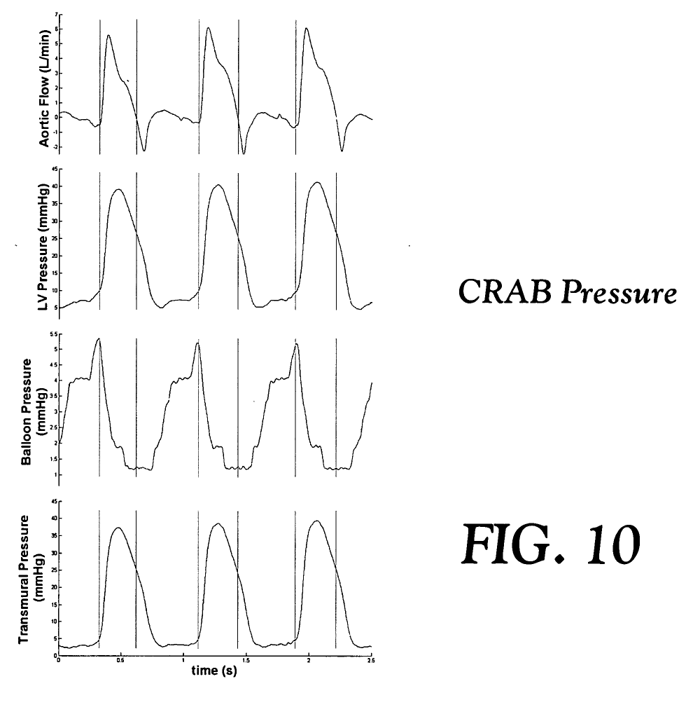

The balloon end diastolic, or CRAB, pressure was noted for varying volumes of fluid instilled in the balloon and graphed as shown in FIG. 9. As shown in FIG. 10, balloon pressure was determined to peak at end diastole and rapidly fall during systole. Balloon pressure was found to rise during ventricular filling before peaking at end diastole (FIG. 10).

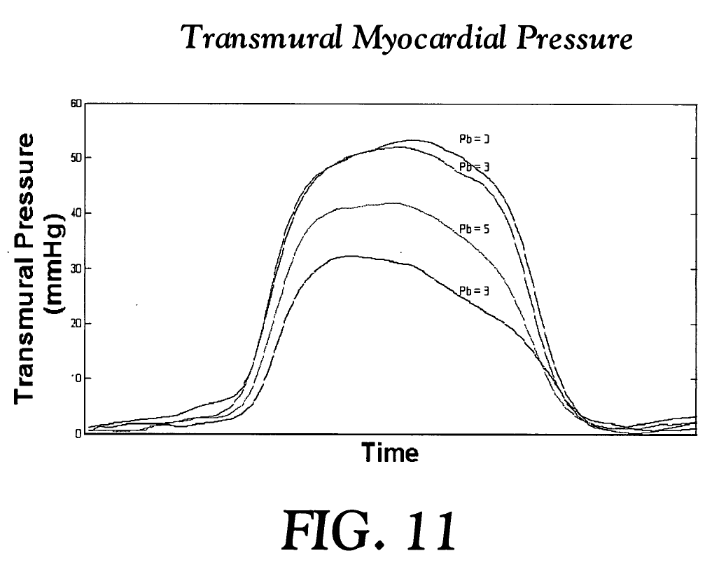

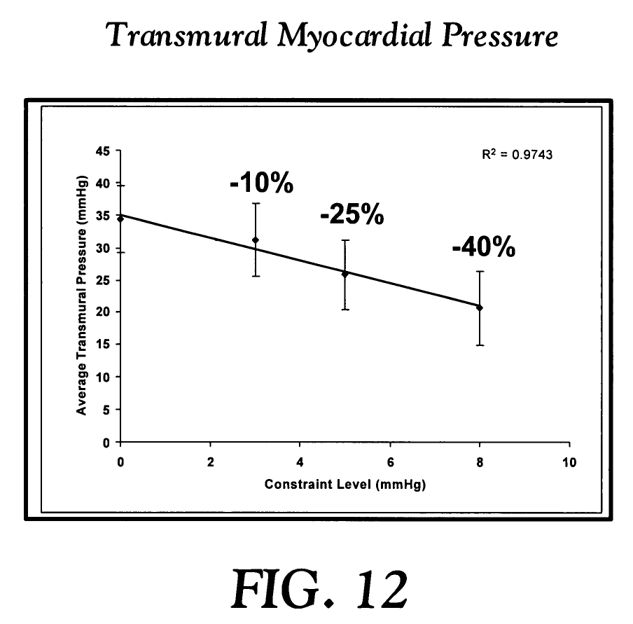

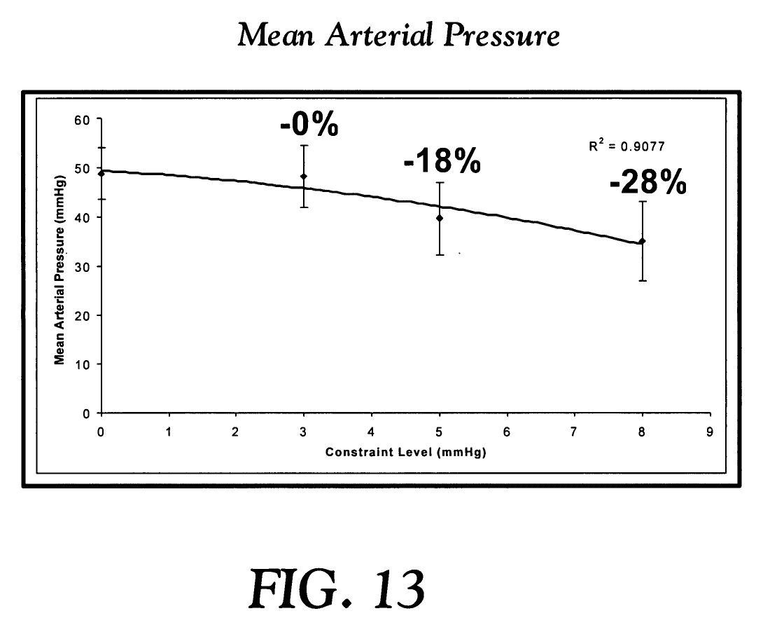

Balloon pressure was varied, and the resulting transmural pressure measured for each pressure point over the length of the cardiac cycle. Transmural myocardial pressure was found to decrease over time throughout the cardiac cycle when the cardiac restraint device was deployed around the ventricles of the heart (p<0.001) (FIGS. 11 and 12). As can be seen in FIGS. 11 and 12, increasing balloon pressure was found to decrease transmural pressure. No significant change in mean arterial pressure was found at low constraint levels (FIG. 13).

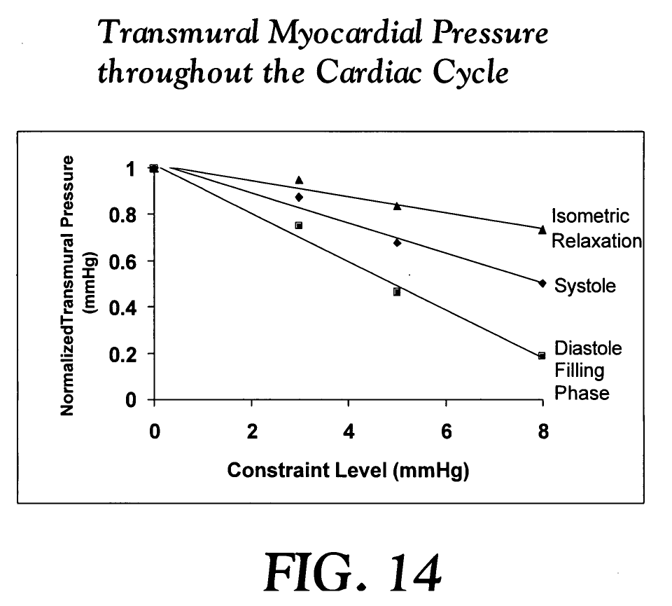

Transmural pressure was determined and normalized for varying constraint levels during each of the following phases of the cardiac cycle: isometric relaxation, systole, and diastole filling phase (FIG. 14). Transmural pressure was found to decrease in all phases of the cardiac cycle with increasing balloon pressure, with the greatest decrease observed during the filling phase of the diastole (FIG. 14).

Thus at low constraint levels an exemplary cardiac restraint device effectively decreases transmural myocardial pressure without significantly affecting arterial pressure. A constraint fluid pressure at end diastole in the range of about 1 mm Hg to about 10 mm Hg may be desired as pressures in this range lower transmural pressure while maintaining arterial pressure in a physiologic range. A constraint fluid pressure at end diastole in the range of about 3 mm Hg to about 8 mm Hg may be desired as pressures in this range also lower transmural pressure while maintaining arterial pressure in a physiologic range. A constraint fluid pressure of about 3 mm Hg, about 5 mm Hg, or about 8 mm Hg may also be desirable for reducing transmural myocardial pressure as these pressures lower transmural pressure while maintaining arterial pressure in a physiologic range.

I claim:

1. A cardiac restraint device, comprising:
a sac, having:
an inner wall;
an outer wall in fluid-tight seal with the inner wall;
an unpartitioned single chamber enclosed between the walls; and
a port in fluid communication with the chamber and accessible from outside the device, to permit instillation of fluid into the chamber and to permit measurement of the fluid pressure; and
a jacket surrounding the sac and affixable thereto;
wherein:
the inner wall:
defines a cavity so sized and shaped as to receive the left and right ventricles of a heart; and
is deformable in response to the instillation of a fluid into the chamber;
the jacket is so nondeformable as not to expand when a fluid is introduced into the chamber;
the device is so sized and shaped that the inner wall engages the outer surface of the ventricles when positioned around the heart; and
the inner wall, when the chamber is instilled with fluid, contacts substantially all of the outer surface of the ventricles.

2. A cardiac restraint device as defined by claim 1, wherein the jacket comprises a fabric.

3. A cardiac restraint device as defined by claim 1, further comprising a tube in fluid communication with the port.

4. A cardiac restraint device as defined by claim 3, wherein the tube extends from the port to a subcutaneous position.

5. A cardiac restraint device as defined by claim 1, wherein the device delivers a pressure to the cavity in the range from about 1 mmHg to about 100 mm Hg when the chamber is filled with fluid.

6. A cardiac restraint device as defined by claim 1, wherein the outer wall is continuous with the inner wall.

7. A cardiac restraint device as defined by claim 1 wherein the fluid pressure is in the range of about 1 mm Hg to about 25 mm Hg.

8. A cardiac restraint device as defined by claim 1 wherein the fluid pressure is in the range of about 1 mm Hg to about 10 mm Hg.

9. A cardiac restraint device as defined by claim 7 wherein the fluid pressure is in the range of about 3 mm Hg to about 8 mm Hg.

10. A cardiac restraint device as defined by claim 1 wherein the fluid pressure is in the range of about 10 mm Hg to about 25 mm Hg.

11. A cardiac restraint device as defined by claim 1 wherein the fluid pressure is in the range of about 20 mm Hg to about 25 mm Hg.

12. A cardiac restraint device, comprising:
a sac, having:
an inner wall;
an outer wall in fluid-tight seal with the inner wall;
an unpartitioned single chamber enclosed between the walls; and
a port in fluid communication with the chamber and accessible from outside the device, to permit instillation of fluid into the chamber and to permit measurement of the fluid pressure;
wherein:
the inner wall:
defines a cavity so sized and shaped as to receive the left and right ventricles of a heart; and
is deformable in response to the instillation of a fluid into the chamber;
the outer wall is so nondefornable as not to expand when a fluid is introduced into the chamber;
the device is so sized and shaped that the inner wall engages the outer surface of the ventricles when positioned around the heart; and
the inner wall, when the chamber is instilled with fluid, contacts substantially all of the outer surface of the ventricles.

13. A method of cardiac restraint, comprising:
fitting a device as defined by claim 1 around a heart, so that the left and right ventricles of the heart occupy the cavity;
affixing the device to the heart; and
instilling fluid into the chamber of the device to exert pressure on the heart;
thereby restraining the heart.

14. A method of cardiac restraint as defined by claim 13, further comprising adjusting the amount of fluid in the chamber to control the pressure of the fluid.

15. A method of cardiac restraint as defined by claim 13, further comprising measuring the pressure in the chamber.

16. A method of cardiac restraint as defined by claim 15, further comprising so adjusting the amount of fluid in the chamber in response to the measured pressure as to achieve a desired pressure.

17. A method of cardiac restraint as defined by claim 16, wherein the desired pressure is in the range of about 1 mm Hg to about 25 mm Hg at end diastole.

18. A method of cardiac restraint as defined by claim 16, wherein the desired pressure is in the range of about 1 mm Hg to about 10 mm Hg at end diastole.

19. A method of cardiac restraint as defined by claim 16, wherein the desired pressure is in the range of about 3 mm Hg to about 8 mm Hg at end diastole.

20. A method of cardiac restraint as defined by claim 16, wherein the desired pressure is in the range of about 10 mm Hg to about 25 mm Hg at end diastole.
